(19) United States
(12) Patent Application Publication
(10) Pub. No.: US 2026/0337186 A1
(43) Pub. Date: Oct. 29, 2026

(54) SUBSTITUTED 3-METHYLBENZO[D]THIAZOL-3-IUM COMPOUNDS AND USE THEREOF

(71) Applicants: COUNCIL OF SCIENTIFIC & INDUSTRIAL RESEARCH, New Delhi (IN); BIOTECH DESK PVT. LTD., Hyderabad (IN)

(72) Inventors: Atul GOEL, Lucknow (IN); Sajiya PARVEEN, Lucknow (IN); Kundan Singh RAWAT, Lucknow (IN); Shradha GOENKA, Lucknow (IN)

(73) Assignee: COUNCIL OF SCIENTIFIC & INDUSTRIAL RESEARCH, New Delhi, DL (IN)

(21) Appl. No.: 18/993,698

(22) PCT Filed: Jul. 12, 2023
(86) PCT No.: PCT/IN2023/050682

§ 371 Date: Jan. 13, 2025

(30) Foreign Application Priority Data
Jul. 12, 2022 (IN) 202211040311

(51) Int. Cl. C07D 417/06; C07D 417/14; C12Q 1/6806; C12Q 1/686

(52) U.S. Cl. CPC C07D 417/06; C07D 417/14; C12Q 1/6806; C12Q 1/686

(57) ABSTRACT

The present invention relates to substituted 3-methylbenzo[d]thiazol-3-ium compounds, their nucleic acid conjugates, complexes, salts which are potentially useful for cell imaging applications, diagnostics, fluorescent tags, pharmaceuticals and other useful molecular biology applications, and a process of preparation thereof. More particularly, the present invention relates to 3-methyl-2-((2-substituted-1-phenylquinolin-4(1H)ylidene)methyl)-benzo[d]thiazol-3-ium compounds, processes for preparing said compounds and their uses for detection of nucleic acids in fluorescence-based imaging and analysis. Specifically, the present invention pertains to a new, dual-function DNA gel stain Compound 1, which serves as an effective nucleic acid stain, demonstrating high sensitivity and affinity towards various nucleic acids, including genomic DNA, PCR products, plasmids, and RNA. Additionally, Compound 1 enhances Real-Time Polymerase Chain Reaction processes by providing improved Relative Fluorescence Units values and enabling the amplification of larger targets. Uniquely, Compound 1 does not necessitate any special or hazardous waste disposal procedures, making it a safer and environmentally sustainable choice for laboratories.

### FIELD OF THE INVENTION

The present invention relates to substituted 3-methylbenzo[d]thiazol-3-ium compounds of the general formula I and their nucleic acid conjugates/complexes, salts which are potentially useful in chemical and biological sciences such as cell imaging applications, diagnostics, fluorescent tags, pharmaceuticals and other useful molecular biology applications, and a process of preparing said new compounds. More particularly, the present invention relates to 3-methyl-2-((2-substituted-1-substituted/unsubstitued-phenylquinolin-4(1H)ylidene)methyl)benzo[d]thiazol-3-ium compounds, processes for preparing the said compounds and their uses for detection of nucleic acids in fluorescence-based imaging and/or analysis. More specifically, the invention relates to the field of nucleic acid detection including genomic DNA, PCR products, plasmids, and RNA and visualization in gel electrophoresis and Real-Time Polymerase Chain Reaction (RT-PCR) processes.

### BACKGROUND OF THE INVENTION

U.S. Pat. No. 5,658,751 (Stephen et al.) reported the synthesis of 2-((2-chloro-1-phenylquinolin-4(1H)-ylidene)methyl)-3-methylbenzo[d]thiazol-3-ium in three steps. In step-I, inventors used 4-methylquinolin-2(1H)-one and iodobenzene in the presence of copper powder and K₂CO₃, the reaction was completed in 48 h and reported 54% yield of the precursor A (4-methyl-1-phenylquinolin-2(1H)-one) which was purified by silica gel column chromatography. In step-II, the precursor B (2-chloro-4-methyl-1-phenylquinolin-1-ium) was synthesized by using DMF and POCl₃ which was further reacted with 3-methyl-2-methylthiobenzothiazolium tosylate (Step-III) to afford the 2-((2-chloro-1-phenylquinolin-4(1H)-ylidene)methyl)-3-methylbenzo[d]thiazol-3-ium in situ as intermediate compound.

Itami et al. have also reported the synthesis of 2-((2-(diethylamino)-1-phenylquinolin-4(1H)-ylidene)methyl)-3-methylbenzo[d]thiazol-3-ium in only 8% without isolating 2-((2-chloro-1-phenylquinolin-4(1H)-ylidene)methyl)-3-methylbenzo[d]thiazol-3-ium (Chem. Asian J. 2017, 12, 233-238).

Moreover, the field of molecular biology and genetic diagnostics has greatly benefited from the advent of DNA gel staining and Real-Time Polymerase Chain Reaction (RT-PCR) technologies. These tools have enabled us to visualize and analyze genetic material in unprecedented detail, facilitating advances in research, healthcare, forensics, and numerous other fields. However, as with any technology, these techniques are not without their limitations and drawbacks.

One significant challenge has been the issue of safety and environmental impact. Traditional nucleic acid stains like ethidium bromide, despite their high sensitivity and effectiveness, are mutagenic. This means that they pose a risk to human health due to their potential to cause genetic mutations, which can lead to serious health problems including cancer. Additionally, due to their hazardous nature, these substances require careful handling and disposal, further increasing the risks associated with their use.

In response to these concerns, safer alternatives such as SYBR Safe have been developed and adopted (U.S. Pat. No. 5,436,134A). SYBR Safe, being non-mutagenic, greatly reduces the health risks associated with DNA staining. Moreover, it offers high sensitivity, making it a valuable tool for visualizing nucleic acids in gel electrophoresis. However, this advancement is not without its shortcomings. Despite the lower toxicity, SYBR Safe and similar dyes still require special disposal procedures, contributing to the operational complexity and cost in laboratories. The environmental implications of these disposal procedures are significant, adding to the overall environmental footprint of research and diagnostic activities in molecular biology.

Furthermore, these newer dyes have primarily found utility in DNA staining. Their use in other important applications such as RT-PCR has been less prominent. The ability to use a single, safe dye across multiple applications would offer significant practical advantages, reducing the need for multiple reagents and simplifying laboratory procedures.

The challenge extends beyond safety and versatility. Another critical limitation with existing methods is the difficulty in amplifying larger targets in RT-PCR. This issue restricts the range of genetic sequences that can be efficiently amplified and detected. The ability to effectively amplify larger targets would unlock new possibilities in genetic research and diagnostics, enabling the study and detection of a broader range of genetic material.

In light of these challenges, the need for a more versatile, safe, and efficient nucleic acid stain becomes evident. This ideal stain would offer high sensitivity and safety, similar to SYBR Safe, while also being environmentally friendly by not necessitating any special disposal procedures. Furthermore, this stain should offer broader utility, applicable not just to DNA staining in gel electrophoresis, but also to enhancing the efficiency and scope of RT-PCR, including the amplification of larger targets.

In the present invention, substituted 3-methylbenzo[d]thiazol-3-ium compounds are designed with these considerations in mind. It aims to address the limitations of existing dyes, offering a more versatile, safe, and environmentally friendly solution for nucleic acid visualization and RT-PCR enhancement. The present invention provides substituted 3-methylbenzo[d]thiazol-3-ium compounds for nucleic acid staining and visualization in molecular biology and genetic diagnostic applications, addressing the limitations of current technologies. In addition to it the invention provides a new high yield process for the preparation of the said compounds.

### OBJECTIVE OF THE INVENTION

The main object of the present invention is to provide substituted 3-methylbenzo[d]thiazol-3-ium compounds having general formula I for nucleic acid staining and visualization in molecular biology and genetic diagnostic applications, addressing the limitations of current technologies. In addition to it the invention provides a new high yield process for the preparation of 3-methyl-2-((2-substituted-1-phenylquinolin-4(1H)-ylidene)methyl)benzo[d]thiazol-3-ium compounds having general formula I.

Another key object of this invention is to ensure high sensitivity in nucleic acid detection, equivalent or superior to that of existing dyes like SYBR Safe. This ensures that even small quantities of nucleic acids can be effectively stained and visualized, enhancing the accuracy and reliability of subsequent analysis.

A further object of the invention is to enhance RT-PCR processes. The compounds of present invention as DNA Gel Stain are formulated to not only serve as a nucleic acid stain but also to improve the sensitivity and scope of RT-PCR applications. This includes the amplification of larger targets, which remains a challenge with existing master mixes.

An additional object of this invention is to provide a safe solution for users and the environment. Our DNA Gel Stain does not require special or hazardous waste disposal procedures, contrasting with many existing dyes. This aligns with global trends towards safer and more environmentally friendly laboratory practices, contributing to sustainable research and diagnostic activities.

The ultimate object of this invention is to provide a versatile, safe, and efficient tool for molecular biology and genetic diagnostics. By addressing the limitations of current technologies, Our DNA Gel Stain aims to simplify laboratory procedures, enhance research capabilities, and facilitate accurate and efficient genetic analysis, all while prioritizing user safety and environmental sustainability.

### SUMMARY OF THE INVENTION

Accordingly, the present invention relates to preparation of substituted 3-methylbenzo[d]thiazol-3-ium compounds as nucleic acid staining probes having general formula I.

In an embodiment, the present invention provides a compound of general formula I, nucleic acid conjugates, complexes and salts thereof,

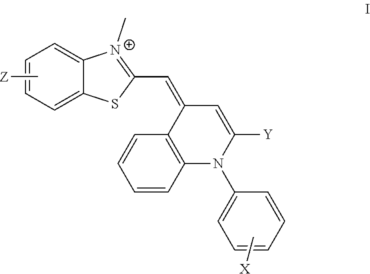

wherein;
X, Z are independently selected from the group consisting of hydrogen, hydroxy, alkyl (C1-C5), alkoxy (OC1-OC5), amine and halogen;
Y is independently selected from the group consisting of

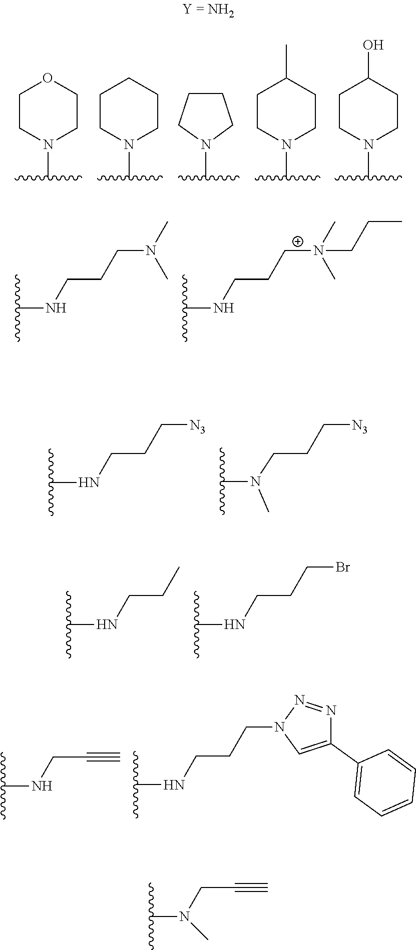

In a preferred embodiment of the present invention the compounds are selected from the group consisting of:
i. 3-methyl-2-((2-morpholino-1-phenylquinolin-4(1H)-ylidene)methyl)benzo[d]thiazol-3-ium;
ii. 2-((2-((3-azidopropyl)amino)-1-phenylquinolin-4(1H)-ylidene)methyl)-3-methylbenzo[d]thiazol-3-ium;
iii. 2-((2-((3-(dimethylamino)propyl)amino)-1-phenylquinolin-4(1H)-ylidene)methyl)-3-methylbenzo[d]thiazol-3-ium;
iv. 2-((2-((3-azidopropyl)(methyl)amino)-1-phenylquinolin-4(1H)-ylidene)methyl)-3-methylbenzo[d]thiazol-3-ium;
v. 3-methyl-2-((1-phenyl-2-(propylamino)quinolin-4(1-ylidene)methyl)benzo[d]thiazol-3-ium.
vi. 3-methyl-2-((2-(methyl(prop-2-yn-1-yl)amino)-1-phenylquinolin-4(1H)-ylidene)methyl)benzo[d]thiazol-3-ium;
vii. 3-methyl-2-((1-phenyl-2-(prop-2-yn-1-ylamino)quinolin-4(1H)-ylidene)methyl)benzo[d]thiazol-3-ium;
viii. 2-((2-((1-(dimethyl(propyl)-14-azaneyl)propan-1-ylium-3-yl)amino)-1-phenylquinolin-4(1H)-ylidene)methyl)-3-methylbenzo[d]thiazol-3-ium;
ix. 2-((2-amino-1-phenylquinolin-4(1H)-ylidene)methyl)-3-methylbenzo[d]thiazol-3-ium.
x. 2-((2-(4-hydroxypiperidin-1-yl)-1-phenylquinolin-4(1H)-ylidene)methyl)-3-methylbenzo[d]thiazol-3-ium;
xi. 3-methyl-2-((1-phenyl-2-(piperidin-1-yl)quinolin-4(1H)-ylidene)methyl)benzo[d]thiazol-3-ium;
xii. 3-methyl-2-((1-phenyl-2-((3-(4-phenyl-1H-1,2,3-triazol-1-yl)propyl)amino)quinolin-4(1H)-ylidene)methyl)benzo[d]thiazol-3-ium;
xiii. 5-hydroxy-3-methyl-2-((2-morpholino-1-phenylquinolin-4(1H)-ylidene)methyl)benzo[d]thiazol-3-ium;
xiv. 3,5-dimethyl-2-((2-morpholino-1-phenylquinolin-4(1H)-ylidene)methyl)benzo[d]thiazol-3-ium;
xv. 4-chloro-3-methyl-2-((2-morpholino-1-phenylquinolin-4(1H)-ylidene)methyl)benzo[d]thiazol-3-ium;
xvi. 7-chloro-3-methyl-2-((2-morpholino-1-phenylquinolin-4(1H)-ylidene)methyl)benzo[d]thiazol-3-ium; and
xvii. 6-chloro-3-methyl-2-((2-morpholino-1-phenylquinolin-4(1H)-ylidene)methyl)benzo[d]thiazol-3-ium.

To another embodiment of the present invention provides a process for the preparation of compound of general formula I,

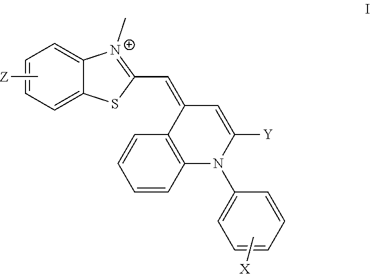

wherein;
X, Z are independently selected from the group consisting of hydrogen, hydroxy, alkyl (C1-C5), alkoxy (OC1-OC5), amine and halogen;
Y is independently selected from the group consisting of

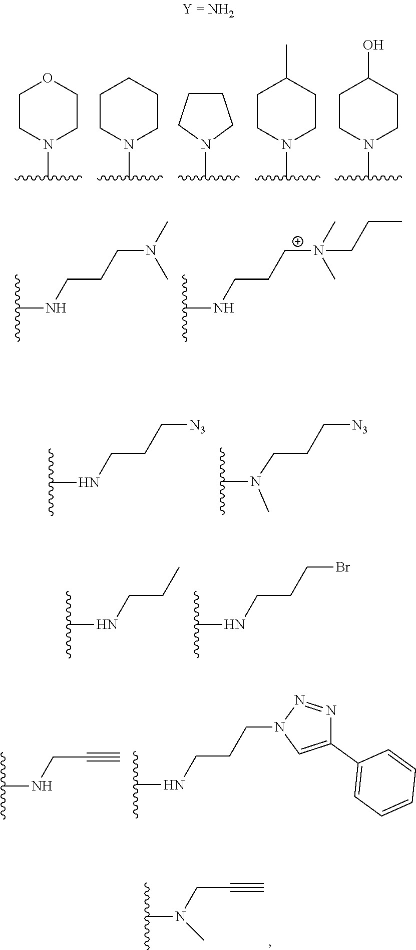

comprising the steps of:
a. reacting a mixture of 4-methylquinolin-2(1H)-one (A) and substituted diphenyliodonium triflate (B) in the presence of copper salt and a base cesium carbonate in presence of an organic solvent under room temperature to reflux condition to form a compound of formula (C);

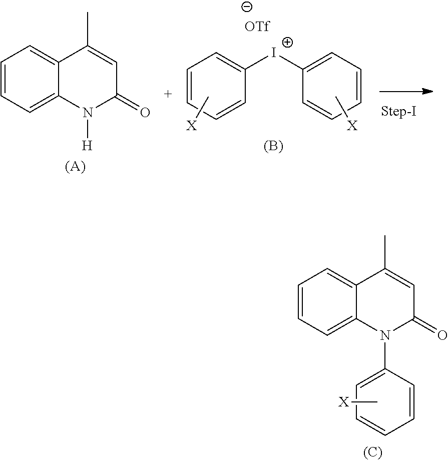

b. reacting a cooled mixture of the compound (C) and substituted 3-methyl-2-(methylthio)benzo[d]thiazol-3-ium (D), in the presence of N,N-diisopropylethylamine in presence of an organic solvent followed by addition of trimethysilyl trifluoromethanesulfonate dropwise to the solution which was then refluxed for 30 min to 24 hr to form a compound of formula (E);

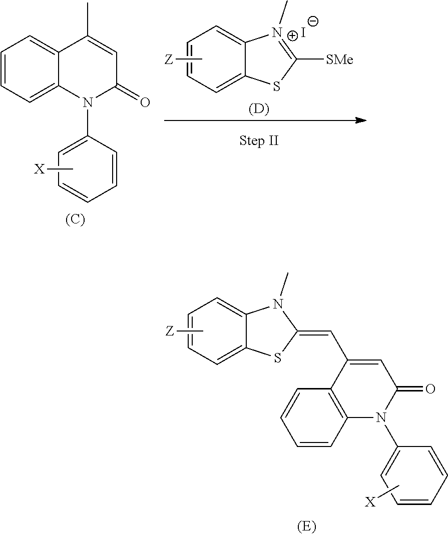

c. reacting the compound (E) in dry dichloromethane with a solution of POCl₃ under reflux to form a compound of formula (F)
   and

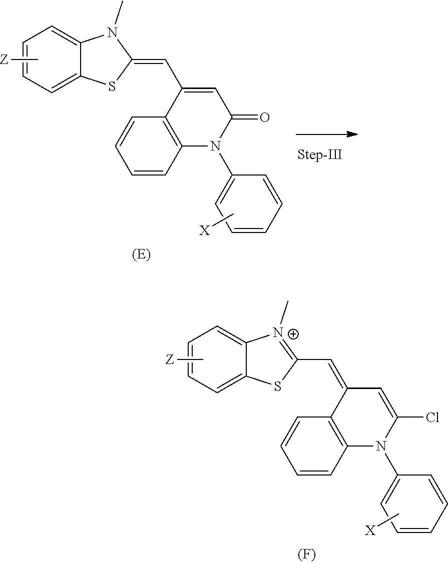

d. reacting the compound (F) with substituted amines (YH) in presence of an organic solvent

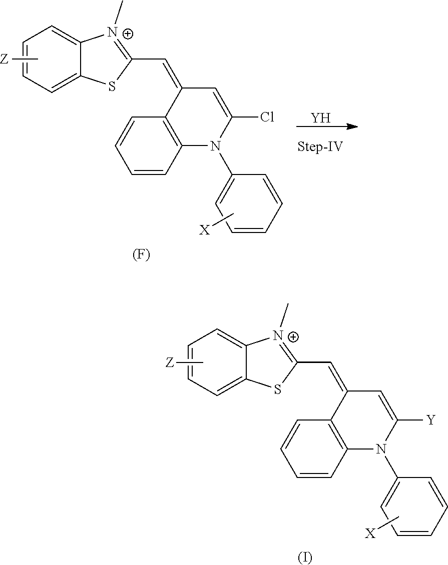

and isolating the compound of general formula I from the reaction mixture and purifying by washing with organic solvents or by chromatography.

In an embodiment of the present invention, the organic solvent is selected from the group consisting of toluene, dichloromethane, dichloroethane, CH₃CN, dimethylsulphoxide, dimethylformamide, water, methanol, ethyl acetate, hexane and tetrahydrofuran.

In an embodiment the present invention provides a compound of general formula I, useful for analyzing nucleic acids (DNA, RNA), and other biological substances of diagnostic importance; useful for development of diagnostic kit for detection of substances, hormones, pathogenic microorganisms, viruses, antibodies, enzymes and nucleic acids, particularly those implicated in disease states; and useful for the preparation of fluorescent probes, tags, markers, diagnostics, ion sensor, pharmaceuticals for detecting/trapping ions in fluorescence-based imaging and/or analysis of cells, biological fluids, chemical mixture and/or other useful applications.

The present invention provides a kit for staining nucleic acids, comprising the compound as claimed in claim 1, for detection of substances, hormones, pathogenic microorganisms, viruses, antibodies, enzymes and nucleic acids.

The present invention provides a kit for enhancing RT-PCR, comprising the compound as claimed in claim 1 and PCR Master mixes consisting of PCR buffer, dNTPs, TaqPolymerase, glycerol and water.

The present invention provides a nucleic acid gel stain, comprising the compound as claimed in claim 1, which exhibits fluorescence in the presence of DNA/RNA.

In a preferred embodiment of the present invention the compound of general formula I, is a nucleic acid stain exhibiting dual functionality in nucleic acid visualization and enhancing Real-Time Polymerase Chain Reaction (RT-PCR) processes, enhanced sensitivity in RT-PCR processes and amplification of larger targets in RT-PCR processes, thereby expanding the range of sequences that can be efficiently amplified and detected; the compound exhibits affinity towards various types of nucleic acids, including but not limited to genomic DNA, PCR products, plasmids, and RNA; and the compound provides clear visualization of nucleic acids under exposure to blue light or UV excitation.

In an embodiment of the present invention the stain demonstrates sensitivity comparable or superior to that of SYBR Safe, facilitating effective staining and visualization of small quantities of nucleic acids.

In an embodiment of the present invention the stain contributes to enhanced sensitivity in RT-PCR processes by providing improved Relative Fluorescence Units (RFU) values compared to other dyes such as SYBR Green.

In an embodiment of the present invention the stain facilitates the amplification of larger targets in RT-PCR processes, thereby expanding the range of sequences that can be efficiently amplified and detected.

In an embodiment of the present invention the stain does not necessitate any special or hazardous waste disposal procedures as required for ethidium bromide, contributing to safer laboratory practices and environmental sustainability.

The present invention provides a method of staining nucleic acids using the compound of general formula I, comprising the steps of: (a) mixing a sample containing nucleic acids with the compounds of Claims 1 and 2; and (b) exposing said sample to blue light or UV excitation to visualize the nucleic acids.

The present invention provides a method for enhancing RT-PCR processes using the compound of general formula I, comprising the steps of: (a) incorporating the compounds of Claims 1 and 2 into an RT-PCR master mix; and (b) performing RT-PCR, resulting in enhanced sensitivity and the ability to amplify larger targets.

### BRIEF DESCRIPTION OF THE DRAWINGS

The present invention may be more clearly understood by reference to the following Tables and Figures:

FIG. 1: Illustrates emission spectra of 3-methyl-2-((2-morpholino-1-phenylquinolin-4(1H)-ylidene)methyl)benzo[d]thiazol-3-ium in PBS Buffer with different concentration of 1 Kb DNA.

FIG. 2: Illustrates emission spectra of 2-((2-((3-azidopropyl)amino)-1-phenylquinolin-4(1H)-ylidene)methyl)-3-methylbenzo[d]thiazol-3-ium in PBS Buffer with different concentration of 1 Kb DNA.

FIG. 3: Illustrates emission spectra of 2-((2-((3-(dimethylamino)propyl)amino)-1-phenylquinolin-4(1H)-ylidene)methyl)-3-methylbenzo[d]thiazol-3-ium in PBS Buffer with different concentration of 1 Kb DNA.

FIG. 4: Illustrates emission spectra of 2-((2-((3-azidopropyl)(methyl)amino)-1-phenylquinolin-4(1H)-ylidene)methyl)-3-methylbenzo[d]thiazol-3-ium in PBS Buffer with different concentration of 1 Kb DNA.

FIG. 5: Illustrates emission spectra of 3-methyl-2-((1-phenyl-2-(propylamino)quinolin-4(1-ylidene)methyl)benzo[d]thiazol-3-ium in PBS Buffer with different concentration of 1 Kb DNA.

FIG. 6: Illustrates emission spectra of 3-methyl-2-((2-(methyl(prop-2-yn-1-yl)amino)-1-phenylquinolin-4(1H)-ylidene)methyl)benzo[d]thiazol-3-ium in PBS Buffer with different concentration of 1 Kb DNA.

FIG. 7: Illustrates emission spectra of 3-methyl-2-((1-phenyl-2-(prop-2-yn-1-ylamino)quinolin-4(1H)-ylidene)methyl)benzo[d]thiazol-3-ium with different concentration of 1 Kb DNA.

FIG. 8: Illustrates emission spectra of 2-((2-((1-(dimethyl(propyl)-14-azaneyl)propan-1-ylium-3-yl)amino)-1-phenylquinolin-4(1H)-ylidene)methyl)-3-methylbenzo[d]thiazol-3-ium in PBS Buffer with different concentration of 1 Kb DNA.

FIG. 9: Illustrates emission spectra of 2-((2-amino-1-phenylquinolin-4(1H)-ylidene)methyl)-3-methylbenzo[d]thiazol-3-ium in PBS Buffer with different concentration of 1 Kb DNA.

FIG. 10: Illustrates emission spectra of 2-((2-(4-hydroxypiperidin-1-yl)-1-phenylquinolin-4(1H)-ylidene)methyl)-3-methylbenzo[d]thiazol-3-ium in PBS Buffer with different concentration of 1 Kb DNA.

FIG. 11: Illustrates emission spectra of 3-methyl-2-((1-phenyl-2-(piperidin-1-yl)quinolin-4(1H)-ylidene)methyl)benzo[d]thiazol-3-ium in PBS Buffer with different concentration of 1 Kb DNA.

FIG. 12: Illustrates emission spectra of 3-methyl-2-((1-phenyl-2-((3-(4-phenyl-1H-1,2,3-triazol-1-yl)propyl)amino)quinolin-4(1H)-ylidene)methyl)benzo[d]thiazol-3-ium in PBS Buffer with different concentration of 1 Kb DNA.

FIG. 13: Illustrate the absorption and emission spectra of the compound 1 in the presence of DNA FIG. 14: Illustrate the Cytotoxicity Study of the Compound 1. Six bacterial plates are displayed, each treated with different concentrations of Compound 1 dissolved in DMSO:DMSO only (control), 10 μM Compound 1, 50 μM Compound 1, 0.1 mM Compound 1, 0.5 mM Compound 1, and 1 mM Compound 1. The absence of a clear zone in all plates indicates that Compound 1, even at the highest concentration tested, does not inhibit bacterial growth or cause cell death, suggesting its non-cytotoxic nature. These findings further underscore the safety of Compound 1 as a nucleic acid stain in laboratory and clinical applications.

FIG. 15: Illustrate the Sensitivity Test of Compound 1. This figure showcases the sensitivity of Compound 1 as a nucleic acid stain. Different amounts of DNA (1 ng, 10 ng, 100 ng, 200 ng, and 500 ng) were loaded on an agarose gel and stained with Compound 1. The resulting image clearly shows that even as little as 10 ng of DNA can be effectively visualized using Compound 1, highlighting its high sensitivity and effectiveness as a DNA stain. This exceptional sensitivity of Compound 1 makes it a powerful tool for detecting and quantifying even low amounts of DNA in research and diagnostic applications.

FIG. 16: Illustrate the Comparison Study of Compound 1 with Other Nucleic Acid Stains. The figure presents a comparative study of the staining efficiency of Compound 1 DNA Gel Stain with two other commonly used nucleic acid stains. Three 1% agarose gels loaded with the same nucleic acid samples were stained differently: Gel 1 with Ethidium Bromide, Gel 2 with SYBR Safe dye from a third party, and Gel 3 with Compound 1 DNA Gel Stain. The resulting images provide a visual comparison of the staining efficiency, clarity, and sensitivity of the three different dyes, underscoring the performance of Compound 1 DNA Gel Stain in nucleic acid detection and visualization. Lane 1-5 represents the gel staining in different type of DNA (1) 100 bp G2P ladder (Catalogue No. L15), (2) 1 Kb G2P ladder (Catalogue No. L12); (3) PCR purified Product_OmiS gene (849 bp)-200 ng, (4) Plasmid P01-200 ng, (5) Genomic DNA-300 ng

FIG. 17: Illustrate Real-Time PCR results recorded using the BioRAD CFX Opus 96 Dx Real-Time PCR System. Four images are presented together: Image A displays the amplification curve, and its corresponding melting curve is shown in Image B. Additionally, Image C compares the amplification curves of Compound 1 and SybrGreen from Vendor A, while Image D compares their respective melting curves. In the experiment, different DNA input concentrations (1 ng, 0.1 ng, and 0.01 ng) were tested with Compound 1 and SybrGreen from Vendor A. The results demonstrate the enhanced sensitivity of Compound 1, enabling the detection of low quantities of amplified nucleic acids. The improved Relative Fluorescence Units (RFU) values obtained with Compound 1 highlight its higher sensitivity and accuracy compared to SybrGreen from Vendor A. The corresponding melting curves in Image D provide further evidence of Compound 1's advantages. The comparable shape and characteristics of the melting curves indicate the similar performance of Compound 1 and SybrGreen from Vendor A in differentiating and identifying specific DNA sequences. Overall, FIG. 18 highlights the enhanced sensitivity and accuracy of Compound 1 as a dye in real-time PCR, showcasing its advantages over existing dyes. These findings support the potential of Compound 1 for precise and reliable nucleic acid detection and analysis in research and diagnostic applications.

FIG. 18: illustrates the amplification of larger targets using Compound 1 and SybrGreen from Vendor an in real-time PCR. (A). The amplification curves for both dyes are displayed. However, it is notable that the curve obtained for Vendor A's SybrGreen exhibits undesired amplification, likely originating from primer dimer formation, as confirmed by running the PCR product on an agarose gel. Compound 1, on the other hand, demonstrates successful amplification of the larger targets without the presence of primer dimer artifacts. (B). Depicts the melting curve of the same experiment. This result highlights the advantage of Compound 1 in enabling the amplification of larger genetic sequences, overcoming the limitations experienced with Vendor A's SybrGreen. By effectively amplifying larger targets, Compound 1 expands the range of genetic material that can be efficiently detected and analyzed in real-time PCR experiments, contributing to the accuracy and reliability of the results.

FIG. 19: illustrates synthesis compound of formula (I)

FIG. 20(A): illustrates synthesis of compound (C);

FIG. 20(B): illustrates synthesis of compound (D)

FIG. 20(C): illustrates synthesis of compound (E)

FIG. 20(D): illustrates synthesis of compound (F)

FIG. 20(E): illustrates synthesis of compound (I)

### ABBREVIATIONS

DCM Dichloromethane
DCE Dichloroethane
DIPEA Diisopropylethylamine
TEA Triethylamine
DMF Dimethylformamide
DNA Deoxyribonucleic acid
RNA Ribonucleic acid
PCR Polymerase chain reaction

### DETAILED DESCRIPTION OF THE INVENTION

The present invention relates to substituted 3-methylbenzo[d]thiazol-3-ium compounds of the general formula I and their ion complexes, salts which are potentially useful in chemical and biological sciences such as cell imaging applications, diagnostics, fluorescent tags, pharmaceuticals and other useful applications, and a process of preparing said new compounds. More particularly, the present invention relates to 3-methyl-2-((2-substituted-1-phenylquinolin-4(1H)ylidene)methyl)benzo[d]thiazol-3-ium compounds, process for preparing the said compounds and their uses for detection of nucleic acids in fluorescence-based imaging and/or analysis.

The present invention more particularly relates to a compound of formula I:

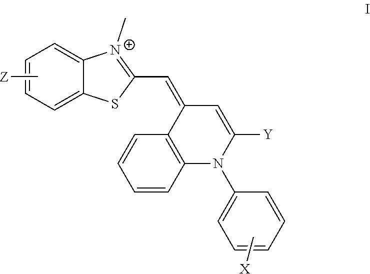

wherein
X, Z are independently selected from the group consisting of hydrogen, hydroxy, alkyl (C1-C5), alkoxy (OC1-OC5), amine and halogen;
Y is independently selected from the group consisting of

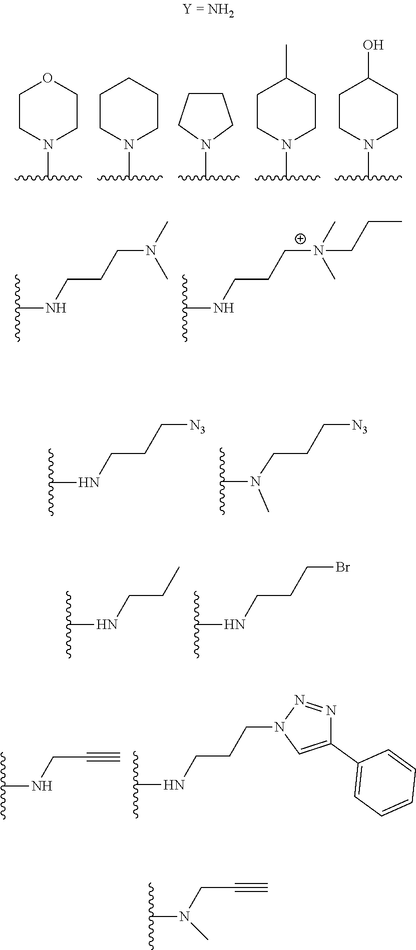

In another embodiment of the invention wherein the representative compounds comprising;
i. 3-methyl-2-((2-morpholino-1-phenylquinolin-4(1H)-ylidene)methyl)benzo[d]thiazol-3-ium.
ii. 2-((2-((3-azidopropyl)amino)-1-phenylquinolin-4(1H)-ylidene)methyl)-3-methylbenzo[d]thiazol-3-ium.
iii. 2-((2-((3-(dimethylamino)propyl)amino)-1-phenylquinolin-4(1H)-ylidene)methyl)-3-methylbenzo[d]thiazol-3-ium.
iv. 2-((2-((3-azidopropyl)(methyl)amino)-1-phenylquinolin-4(1H)-ylidene)methyl)-3-methylbenzo[d]thiazol-3-ium.
v. 3-methyl-2-((1-phenyl-2-(propylamino)quinolin-4(1-ylidene)methyl)benzo[d]thiazol-3-ium.
vi. 3-methyl-2-((2-(methyl(prop-2-yn-1-yl)amino)-1-phenylquinolin-4(1H)-ylidene)methyl)benzo[d]thiazol-3-ium.
vii. 3-methyl-2-((1-phenyl-2-(prop-2-yn-1-ylamino)quinolin-4(1H)-ylidene)methyl)benzo[d]thiazol-3-ium.
viii. 2-((2-((1-(dimethyl(propyl)-14-azaneyl)propan-1-ylium-3-yl)amino)-1-phenylquinolin-4(1H)-ylidene)methyl)-3-methylbenzo[d]thiazol-3-ium.
ix. 2-((2-amino-1-phenylquinolin-4(1H)-ylidene)methyl)-3-methylbenzo[d]thiazol-3-ium.
x. 2-((2-(4-hydroxypiperidin-1-yl)-1-phenylquinolin-4(1H)-ylidene)methyl)-3-methylbenzo[d]thiazol-3-ium
xi. 3-methyl-2-((1-phenyl-2-(piperidin-1-yl)quinolin-4(1H)-ylidene)methyl)benzo[d]thiazol-3-ium
xii. 3-methyl-2-((1-phenyl-2-((3-(4-phenyl-1H-1,2,3-triazol-1-yl)propyl)amino)quinolin-4(1H)-ylidene)methyl)benzo[d]thiazol-3-ium
xiii. 5-hydroxy-3-methyl-2-((2-morpholino-1-phenylquinolin-4(1H)-ylidene)methyl)benzo[d]thiazol-3-ium
xiv. 3,5-dimethyl-2-((2-morpholino-1-phenylquinolin-4(1H)-ylidene)methyl)benzo[d]thiazol-3-ium
xv. 4-chloro-3-methyl-2-((2-morpholino-1-phenylquinolin-4(1H)-ylidene)methyl)benzo[d]thiazol-3-ium
xvi. 7-chloro-3-methyl-2-((2-morpholino-1-phenylquinolin-4(1H)-ylidene)methyl)benzo[d]thiazol-3-ium
xvii. 6-chloro-3-methyl-2-((2-morpholino-1-phenylquinolin-4(1H)-ylidene)methyl)benzo[d]thiazol-3-ium

Another embodiment of the present invention provides a process for the preparation of new 3-methyl-2-((2-substituted-1-phenylquinolin-4(1H)-ylidene)methyl)benzo[d]thiazol-3-ium having the general formula I as shown in scheme 1.

The compound 4-methyl-1-phenylquinolin-2(1H)-one (C) in 80% yield has been synthesized by using the 4-methylquinolin-2(1H)-one and diphenyliodonium trifluoromethanesulfonate in the presence of copper iodide and triethyl amine in toluene at reflux condition for 8 h in step I. In step-IT the 4-methyl-1-phenylquinolin-2(1H)-one (C) was reacted with 3-methyl-2-(methylthio)benzo[d]thiazol-3-ium iodide (D) (prepared by 2-methylthio-benzthiazole and methyl iodide) in the presence of di-isopropyl ethyl amine and trimethylsilyl trifluoro methane sulphonic acid in DCM at 50° C. for 2 h which gave the 4-((3-methylbenzo[d]thiazol-2(3H)-ylidene)methyl)-1-phenylquinolin-2(1H)-one (E) in 75% yield which was purified by washing with water and ethyl acetate and characterized by mass and NMR data. After characterization, 2-chloro-4-((3-methylbenzo[d]thiazol-2(3H)-ylidene)methyl)-1-phenylquinolin-1-ium chloride (F) has been synthesized in step-IT by using DCM and POCl₃ to give 91% yield which was isolated by simple water workup and characterized by mass and NMR data.

In the existing art the compound F has been isolated and stored at room temperature for a long time. In the final step-IV, 2-chloro-4-((3-methylbenzo[d]thiazol-2(3H)-ylidene)methyl)-1-phenylquinolin-1-ium chloride was reacted with primary or secondary amine (YH) in the presence of catalytic amount of DIPEA in DCE at 50° C. and the desired product (I) was obtained in good to excellent yield.

### Synthesis of 3-methyl-2-(methylthio)benzo[d]thiazol-3-ium

A solution of 2-(methylthio)benzo[d]thiazole (1 mL, 5.5 mmol, 1 eq.) in ethanol (10 ml) was added methyl iodide (0.775 mL, 11 mmol, 2 eq.) was refluxed for 24 h and reaction was monitored by taking TLC. After completion, the reaction mixture was cooled to room temperature and solid thus obtained was filtered and washed with diethyl ether to furnish compound 1 as a white solid 900 mg (83%).

### Step-1: Synthesis of 4-methyl-1-phenylquinolin-2(1H)-one

A mixture of 4-methylquinolin-2(1H)-one (500 mg, 3.14 mmol, 1 eq) and diphenyliodonium triflate (3.3 g, 7.8 mmol, 2.5 eq.), CuI (190 mg, 0.62 mmol, 0.2 eq.), caesium carbonate (2 g, 6.2 mmol, 2 eq.) in toluene (10 mL) was reflux for 14 h. The progress of the reaction was monitored by TLC and After completion, the reaction mixture was cooled to room temperature where upon toluene was removed by rotary evaporation. Water was added to the residue and reaction mixture was extracted with DCM. The organic layer was dried over Na₂SO₄ and evaporated under vacuum to give the crude product that was purified by flash column chromatography on silica gel column using 10% ethyl acetate/hexane as eluent to give 620 mg (80%) as a white solid.

### Step-2: Synthesis of 4-((3-methylbenzo[d]thiazol-2(3H)-ylidene)methyl)-1-phenylquinolin-2(1H)-one

To a mixture of 1,2-dihydro-4-methyl-1-phenyl-2-quinolone (1) (500 mg, 2.1 mmol, 1 equiv) 3-methyl-2-methylthiobenzothiazolium iodide (2) (417 mg, 21 mmol, 1 equiv), and N,N-diisopropylethylamine (0.536 mL, 3.1 mmol, 1.5 equiv) in DCM (20 mL) at 0° C. was added trimethysilyl trifluoromethanesulfonate (0.446 mL, 2.1 mmol, 1 equiv) dropwise. After the addition was completed, the solution was refluxed for 1 h and then cooled down to 0° C. Water (110 mL) was added dropwise and the mixture was stirred at 0° C. for 1 h. The aqueous layer was extracted with DCM and the combined organic layers were dried with anhydrous Na2SO4. The solvent was removed and the residue was dried to a constant weight and the compound 3 was purified by silica gel column using 20% ethyl acetate in hexane as eluent to give 470 mg (58%) as an off white solid.

### Step-3: Synthesis of 2-((2-chloro-1-phenylquinolin-4(1H)-ylidene)methyl)-3-methylbenzo[d]thiazol-3-ium

Compound 3 (200 mg, 0.52 mmol, 1 eq.) and POCl3 (250 mg, 1.6 mmol) dissolved in dry dichloromethane (20 mL) and the reaction mixture was reflux for 5 h. After completion of reaction, excess dichloromethane and unreacted POCl3 were removed in vacuum. To this reaction mixture water was added and stirred for 30 min. The red precipitate of compound 4 was obtained after filtration in good yield (190 mg, 91%).

### Step-4: Synthesis of 3-methyl-2-((2-substituted-1-phenylquinolin-4(1H)-ylidene)methyl)benzo[d]thiazol-3-ium

The compound 2-((2-chloro-1-phenylquinolin-4(1H)-ylidene)methyl)-3-methylbenzo[d]thiazol-3-ium (100 mg, 0.25 mmol, 1 equiv) was dissolved in dichloroethane, then primary or secondary amine (1.25 mmol, 5 equiv) were added and the catalytic amount of DIPEA was added and the reaction mixture was stirred at 50° C. for 1-4 h. After concentration under vacuum, the crude product was purified to give red powder in good to excellent yield.

### EXAMPLES

Following examples are given by way of illustration and should not construe the scope of the present invention.

### Example-1

### 3-methyl-2-((2-morpholino-1-phenylquinolin-4(1H)-ylidene) methyl)benzo[d]thiazol-3-ium (1)

The compound 2-chloro-4-((3-methylbenzo[d]thiazol-2(3H)-ylidene)methyl)-1-phenylquinolin-1-ium chloride (100 mg, 0.25 mmol, 1 equiv) was dissolved in dichloroethane, then morpholine (108 mg, 1.25 mmol, 5 equiv) were added and the catalytic amount of DIPEA was added and the reaction mixture was stirred at 50° C. for 6 h. After concentration by the rotary evaporator, the crude product was purified by column chromatography on silica gel 10% DCM/methanol eluent to give red powder 75 mg (yield 66%).

R_{f}=0.54 (chloroform/methanol, 20:1, v/v); mp (chloroform/methanol) 146-148° C.; MS (ESI) 452 [M+H]⁺; HRMS calculated for C₂₈H₂₆N₃OS⁺ [M+H]⁺, 452.1791 found: 452.1786.

### Example-2

### Synthesis of 2-((2-((3-azidopropyl)amino)-1-phenylquinolin-4(1H)-ylidene)methyl)-3-methylbenzo[d]thiazol-3-ium (2)

The compound 2-chloro-4-((3-methylbenzo[d]thiazol-2(3H)-ylidene)methyl)-1-phenylquinolin-1-ium (100 mg, 0.25 mmol, 1 equiv) was dissolved in dichloroethane, then 3-azidopropan-1-amine (50 mg, 0.5 mmol, 2 equiv) were added and the catalytic amount of DIPEA was added and the reaction mixture was stirred at 50° C. for 4 h. After concentration by the rotary evaporator, the crude product was purified by column chromatography on silica gel 10% DCM/methanol eluent to give yellow powder 80 mg (yield 69%).

R_{f}=0.54 (chloroform/methanol, 20:1, v/v); mp (chloroform/methanol) 203-204° C.; MS (ESI) 465 [M]⁺; HRMS calculated for C₂₇H₂₅N₆S⁺ [M]⁺, 465.1856 found: 465.1853.

### Example-3

### 2-((2-((3-(dimethylamino)propyl)amino)-1-phenylquinolin-4(1H)-ylidene)methyl)-3-methylbenzo[d]thiazol-3-ium (3)

The compound 2-chloro-4-((3-methylbenzo[d]thiazol-2(3H)-ylidene)methyl)-1-phenylquinolin-1-ium chloride (100 mg, 0.25 mmol, 1 equiv) was dissolved in dichloroethane, then N1,N1-dimethylpropane-1,3-diamine (50 mg, 0.5 mmol, 2 equiv) were added and the catalytic amount of DIPEA was added and the reaction mixture was stirred at 50° C. for 4 h. After concentration by the rotary evaporator, the crude product was purified by column chromatography on silica gel 10% DCM/methanol eluent to give yellow powder 75 mg (yield 64%). R_{f}=0.56 (chloroform/methanol, 20:1, v/v); mp (chloroform/methanol) 166-167° C.; MS (ESI) 467 [M]⁺; HRMS calculated for C₂₉H₃₁N₄S⁺ [M]⁺, 467.2264 found: 467.2255.

### Example-4

### 2-((2-((3-azidopropyl)(methyl)amino)-1-phenylquinolin-4(1H)-ylidene)methyl)-3-methylbenzo[d]thiazol-3-ium (4)

The compound 2-((2-((3-azidopropyl)amino)-1-phenylquinolin-4(1H)-ylidene)methyl)-3-methylbenzo[d]thiazol-3-ium (50 mg, 0.10 mmol, 1 equiv) was dissolved in DMF, then methyl iodide (30 ul, 0.5 mmol, 2 equiv) were added and the catalytic amount of DIPEA was added and the reaction mixture was stirred at room temperature for 3 h. After completion the reaction mixture was poured into ice cold water and precipitate were filtered out, the crude product was purified by column chromatography on silica gel 10% DCM/methanol eluent to give red powder 40 mg (yield 83%). R_{f}=0.54 (chloroform/methanol, 20:, v/v); mp (chloroform/methanol) 191-193° C.; MS (ESI) 479 [M]⁺; HRMS calculated for C₂₈H₂₇N₆S⁺ [M]⁺, 479.2012 found: 479.2026.

### Example-5

### 3-methyl-2-((1-phenyl-2-(propylamino)quinolin-4(1-ylidene)methyl)benzo[d]thiazol-3-ium (5)

The compound 2-chloro-4-((3-methylbenzo[d]thiazol-2(3H)-ylidene)methyl)-1-phenylquinolin-1-ium chloride (100 mg, 0.25 mmol, 1 equiv) was dissolved in dichloroethane, then propyl amine (50 mg, 0.5 mmol, 2 equiv) were added and the catalytic amount of DIPEA was added and the reaction mixture was stirred at 50° C. for 4 h. After concentration by the rotary evaporator, the crude product was purified by column chromatography on silica gel 10% DCM/methanol eluent to give yellow powder 75 mg (yield 64%). R_{f}=0.56 (chloroform/methanol, 20:1, v/v); mp (chloroform/methanol) 236-238° C.; MS (ESI) 420 [M]⁺; HRMS calculated for C₂₇H₂₂N₃S+ [M]⁺, 420.1529 found: 420.1531.

### Example-6

### 3-methyl-2-((2-(methyl(prop-2-yn-1-yl)amino)-1-phenylquinolin-4(1H)-ylidene)methyl)benzo[d]thiazol-3-ium (6)

The compound 3-methyl-2-((1-phenyl-2-(propylamino)quinolin-4(lylidene)methyl)benzo[d]thiazol-3-ium (100 mg, 0.23 mmol, 1 equiv) was dissolved in DMF and the catalytic amount of DIPEA was added and the reaction mixture was stirred at room temperature for 30 minute, after that methyl iodide (70 ul, 0.49 mmol, 5 equiv) were added. After completion the reaction, the reaction mixture was poured into ice cold water and precipitate was filtered out. The crude product was purified by column chromatography on silica gel 20% DCM/methanol eluent to give yellow powder 75 mg (yield 64%). R_{f}=0.56 (chloroform/methanol, 20:1, v/v); mp (chloroform/methanol) 221-223° C.; MS (ESI) 434 [M]⁺; HRMS calculated for C₂₈H₂₄N₃S⁺ [M]⁺, 434.1685 found: 434.1701.

### Example-7

### 3-methyl-2-((1-phenyl-2-(prop-2-yn-1-ylamino)quinolin-4(1H)-ylidene)methyl)benzo[d]thiazol-3-ium (7)

The compound 2-chloro-4-((3-methylbenzo[d]thiazol-2(3H)-ylidene)methyl)-1-phenylquinolin-1-ium chloride (100 mg, 0.25 mmol, 1 equiv) was dissolved in dichloroethane, then proparzyl amine (50 mg, 0.5 mmol, 2 equiv) were added and the catalytic amount of DIPEA was added and the reaction mixture was stirred at 50° C. for 2 h. After concentration by the rotary evaporator, the crude product was purified by column chromatography on silica gel 10% DCM/methanol eluent to give yellow powder 75 mg (yield 64%). R_{f}=0.56 (chloroform/methanol, 20:2, v/v); mp (chloroform/methanol) 209-210° C.; MS (ESI) 420 [M]⁺; HRMS calculated for C₂₇H₂₂N₃S⁺ [M]⁺, 420.1529 found: 420.1531.

### Example-8

### 2-((2-((1-(dimethyl(propyl)-14-azaneyl)propan-1-ylium-3-yl)amino)-1-phenylquinolin-4(1H)-ylidene)methyl)-3-methylbenzo[d]thiazol-3-ium (8)

The compound 2-((2-((3-(dimethylamino)propyl)amino)-1-phenylquinolin-4(1H)-ylidene)methyl)-3-methylbenzo[d]thiazol-3-ium (50 mg, 0.10 mmol, 1 equiv) was dissolved in DMF, then propyl iodide (50 ul, 0.29 mmol, 5 equiv) were added and the reaction mixture was stirred at 50° C. for 6 h. After completion the reaction the crude was poured into ice cold water and precipitate was filtered out, the crude product was purified by column chromatography on silica gel 20% DCM/methanol eluent to give yellow powder 40 mg (yield 78%). R_{f}=0.25 (chloroform/methanol, 20:2, v/v); mp (Dichloromethane/methanol) 255-257° C.; HRMS calculated for C₃₂H₃₈N₄S²⁺ [M²⁺]m/z=510.2806/2=255.1403; Found: 255.1409.

### Example-9

### 2-((2-amino-1-phenylquinolin-4(1H)-ylidene)methyl)-3-methylbenzo[d]thiazol-3-ium (9)

The compound of 2-chloro-4-((3-methylbenzo[d]thiazol-2(3H)-ylidene)methyl)-1-phenylquinolin-1-ium chloride (100 mg, 0.25 mmol, 1 equiv) was dissolved in DMF and then liquid ammonia were added in excess and the reaction mixture was stirred at 50° C. for 5 h. After completion the reaction the crude was poured into ice cold water and precipitate was filtered out, the crude product was purified by column chromatography on silica gel 10% DCM/methanol eluent to give yellow powder 45 mg (yield 47%). R_{f}=0.56 (chloroform/methanol, 10:2, v/v); mp (chloroform/methanol) 198-199° C.; MS (ESI) 382 [M]⁺; HRMS calculated for C₂₄H₂₀N₃S⁺ [M]⁺, 382.1372 found: 382.1363.

### Example-10

### 2-((2-(4-hydroxypiperidin-1-yl)-1-phenylquinolin-4(1H)-ylidene)methyl)-3-methylbenzo[d]thiazol-3-ium (10)

The compound 2-chloro-4-((3-methylbenzo[d]thiazol-2(3H)-ylidene)methyl)-1-phenylquinolin-1-ium chloride (100 mg, 0.25 mmol, 1 equiv) was dissolved in dichloroethane, then 4-hydroxy piperidine (50 mg, 0.5 mmol, 2 equiv) were added and the catalytic amount of DIPEA was added and the reaction mixture was stirred at 50° C. for 4 h. After concentration by the rotary evaporator, the crude product was purified by column chromatography on silica gel 10% DCM/methanol eluent to give yellow powder 85 mg (yield 78%). R_{f}=0.56 (chloroform/methanol, 20:2, v/v); mp (chloroform/methanol) 222-223° C.; MS (ESI) 466 [M]⁺; HRMS calculated for C₂₉H₂₈N₃OS⁺ [M]⁺, 466.1948 found: 466.1952.

### Example-11

### 3-methyl-2-((1-phenyl-2-(piperidin-1-yl)quinolin-4(1H)-ylidene)methyl)benzo[d]thiazol-3-ium (11)

The compound 2-chloro-4-((3-methylbenzo[d]thiazol-2(3H)-ylidene)methyl)-1-phenylquinolin-1-ium chloride (100 mg, 0.25 mmol, 1 equiv) was dissolved in dichloroethane, then piperidine (100 mg, 0.5 mmol, 4 equiv) were added and the catalytic amount of DIPEA was added and the reaction mixture was stirred at 50° C. for 4 h. After concentration by the rotary evaporator, the crude product was purified by column chromatography on silica gel 10% DCM/methanol eluent to give yellow powder 80 mg (yield 71%). R_{f}=0.56 (chloroform/methanol, 20:2, v/v); mp (chloroform/methanol) 238-237° C.; MS (ESI) 450 [M]⁺; HRMS calculated for C₂₉H₂₈N₃S⁺ [M]⁺, 450.1998 found: 450.1993.

### Example-12

### 3-methyl-2-((1-phenyl-2-((3-(4-phenyl-1H-1,2,3-triazol-1-yl)propyl)amino)quinolin-4(1H)-ylidene)methyl)benzo[d]thiazol-3-ium (12)

The compound 2-((2-((3-azidopropyl)amino)-1-phenylquinolin-4(1H)-ylidene)methyl)-3-methylbenzo[d]thiazol-3-ium compound (100 mg, 0.21 mmol, 1 equiv) and sodium ascorbate (21 mg, 0.10 mmol, 0.5 equiv), CuSO₄·6H₂O (28 mg, 0.10 mmol, 0.5 equiv,), phenyl acetylene (45 uL, 2 equiv) was added respectively in 1:1 butanol water as a solvent at room temperature to 60° C. after completion the reaction butanol water was evaporated by rotary evaporator, the crude was purified by column chromatography on silica gel 10% DCM/methanol eluent to give orange powder 60 mg (yield 51%). R_{f}=0.52 (chloroform/methanol, 20:2, v/v); mp (chloroform/methanol) 233-234° C.; MS (ESI) 567 [M]⁺; HRMS calculated for C₃₅H₃₁N₆S⁺ [M]⁺, 567.2325 found: 567.2327.

### Example-13

### 5-hydroxy-3-methyl-2-((2-morpholino-1-phenylquinolin-4(1H)-ylidene)methyl)benzo[d]thiazol-3-ium (13)

The compound 2-(chloro-1-phenylquinolin-4(1H)-ylidene)methyl)-5-hydroxy-3-methylbenzo[d]thiazol-3-ium (100 mg, 0.25 mmol, 1 equiv) was dissolved in dichloroethane, then morpholine (200 ul, 0.6 mmol, 3 equiv) was added and the catalytic amount of DIPEA was added and the reaction mixture was stirred at 50° C. for 5 h. After concentration by the rotary evaporator, the crude product was purified by column chromatography on silica gel 10% DCM/methanol eluent to give red powder 60 mg (yield 59%).

MS (ESI) 468 [M]⁺; HRMS calculated for C₂₈H₂₆N₃O₂S⁺ [M]⁺, 468.1740 found: 468.1752.

### Example 14

### 3,5-dimethyl-2-((2-morpholino-1-phenylquinolin-4(1H)-ylidene)methyl)benzo[d]thiazol-3-ium (14)

The compound 2-(chloro-1-phenylquinolin-4(1H)-ylidene)methyl)-3,5-dimethylbenzo[d]thiazol-3-ium (100 mg, 0.27 mmol, 1 equiv) was dissolved in dichloroethane, then morpholine (200 ul, 0.7 mmol, 3 equiv) was added and the catalytic amount of DIPEA was added and the reaction mixture was stirred at 50° C. for 5 h. After concentration by the rotary evaporator, the crude product was purified by column chromatography on silica gel 10% DCM/methanol eluent to give orange powder 70 mg (yield 55%).

MS (ESI) 466 [M]⁺; HRMS calculated for C₂₉H₂₈N₃OS⁺ [M]⁺, 466.1948 found: 466.1957.

### Example 15

### 4-chloro-3-methyl-2-((2-morpholino-1-phenylquinolin-4(1H)-ylidene)methyl)benzo[d]thiazol-3-ium (15)

The compound 4-chloro-2-((2-chloro-1-phenylquinolin-4(1H)-ylidene)methyl)-3-methylbenzo[d]thiazol-3-ium (100 mg, 0.21 mmol, 1 equiv) was dissolved in dichloroethane, then morpholine (220 ul, 0.5 mmol, 3 equiv) was added and the catalytic amount of DIPEA was added and the reaction mixture was stirred at 50° C. for 5 h. After concentration by the rotary evaporator, the crude product was purified by column chromatography on silica gel 10% DCM/methanol eluent to give orange powder 60 mg (yield 60%).

MS (ESI) 486 [M]⁺; HRMS calculated for C₂₈H₂₅ClN₃OS⁺ [M]⁺, 486.1401 found: 486.1409.

### Example 16

### 7-chloro-3-methyl-2-((2-morpholino-1-phenylquinolin-4(1H)-ylidene)methyl)benzo[d]thiazol-3-ium (16)

The compound 7-chloro-2-((2-chloro-1-phenylquinolin-4(1H)-ylidene)methyl)-3-methylbenzo[d]thiazol-3-ium (100 mg, 0.21 mmol, 1 equiv) was dissolved in dichloroethane, then morpholine (220 ul, 0.5 mmol, 3 equiv) was added and the catalytic amount of DIPEA was added and the reaction mixture was stirred at 50° C. for 5 h. After concentration by the rotary evaporator, the crude product was purified by column chromatography on silica gel 10% DCM/methanol eluent to give orange powder 60 mg (yield 60%).

R_{f}=0.55 (chloroform/methanol, 20:1, v/v); MS (ESI) 486 [M]⁺; HRMS calculated for C₂₈H₂₅ClN₃OS⁺ [M]⁺, 486.1401 found: 486.1402.

### Example 17

### 6-chloro-3-methyl-2-((2-morpholino-1-phenylquinolin-4(1H)-ylidene)methyl)benzo[d]thiazol-3-ium (17)

The compound 6-chloro-2-((2-chloro-1-phenylquinolin-4(1H)-ylidene)methyl)-3-methylbenzo[d]thiazol-3-ium (100 mg, 0.21 mmol, 1 equiv) was dissolved in dichloroethane, then morpholine (220 ul, 0.5 mmol, 3 equiv) was added and the catalytic amount of DIPEA was added and the reaction mixture was stirred at 50° C. for 5 h. After concentration by the rotary evaporator, the crude product was purified by column chromatography on silica gel 10% DCM/methanol eluent to give orange powder 50 mg (yield 55%).

MS (ESI) 486 [M]⁺; HRMS calculated for C₂₈H₂₅ClN₃OS⁺ [M]⁺, 486.1401 found: 486.1413.

### Photophysical Property of the Compounds of Invention

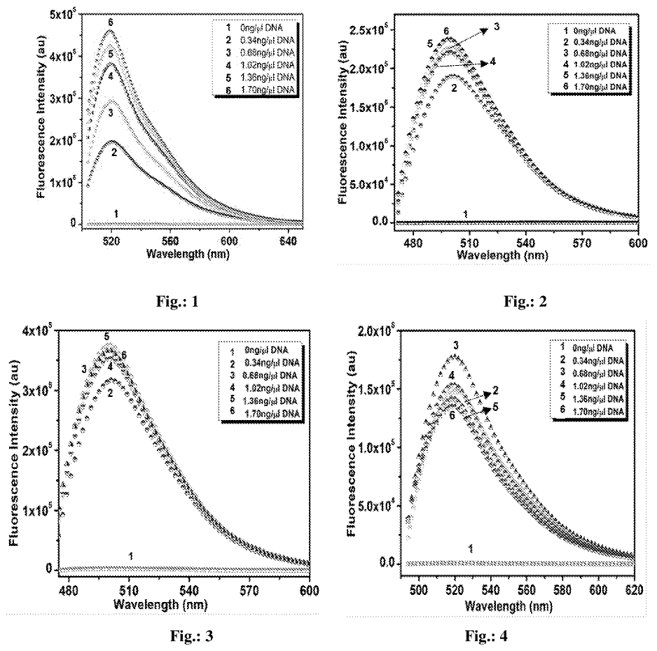

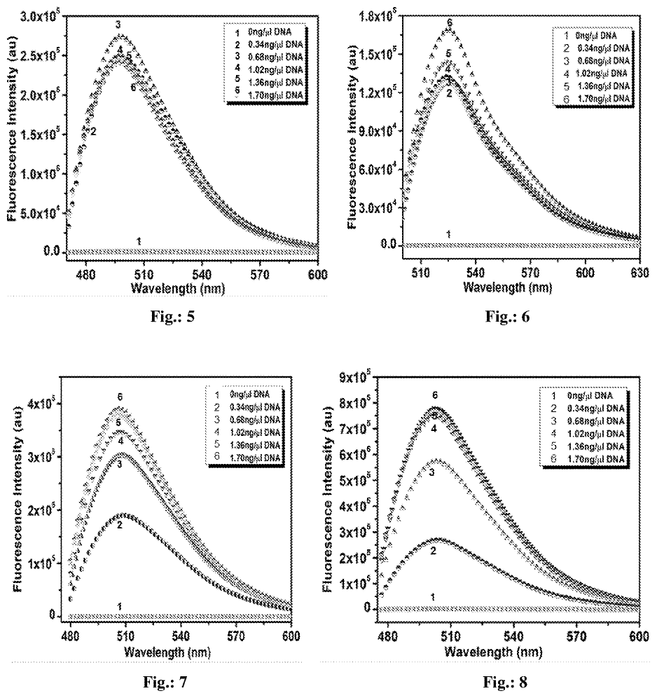

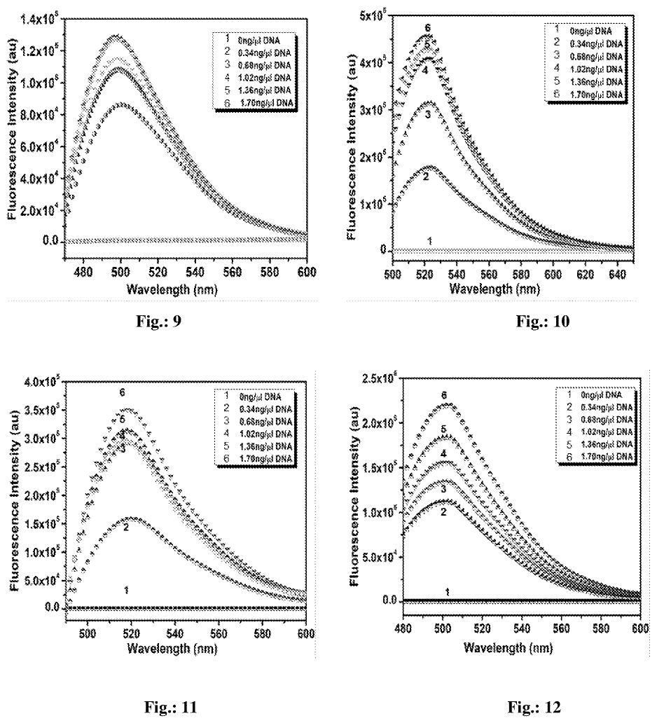

The photophysical properties of all the synthesized compounds were examined by UV-vis and fluorescence techniques. Absorption and emission spectra of 3-methyl-2-((2-substituted-amino-1-phenylquinolin-4(1H)-ylidene)methyl)benzo[d]thiazol-3-ium compounds in PBS Buffer with different concentration of 1 Kb DNA (FIG. 1-12).

TABLE 1
Photophysical studies of the compounds of invention
in the presence of nucleic acid (1 Kb DNA)

| Example | λ_{max, abs} nm | λ_{max, em} with DNA nm |
|---|---|---|
| 1 | 500 | 520 |
| 2 | 462 | 498 |
| 3 | 465 | 497 |
| 4 | 484 | 518 |
| 5 | 461 | 496 |
| 6 | 491 | 523 |
| 7 | 469 | 505 |
| 8 | 467 | 500 |
| 9 | 483 | 498 |
| 10 | 490 | 519 |
| 11 | 480 | 520 |
| 12 | 470 | 497 |

Exemplary, the compound 1 DNA Gel Stain exhibits unique fluorescence properties that are triggered upon binding with nucleic acids, making it a highly sensitive and responsive tool for molecular biology applications.

In its native state, dissolved in DMSO solution, Compound 1 is non-fluorescent. This property prevents any background fluorescence, facilitating a clean and clear readout when the dye is used for staining nucleic acids. Upon binding with nucleic acids such as DNA or RNA, Compound 1 undergoes a change that results in the emission of green fluorescence. This fluorescence change is indicative of the presence and quantity of nucleic acids, enabling accurate visualization and quantification.

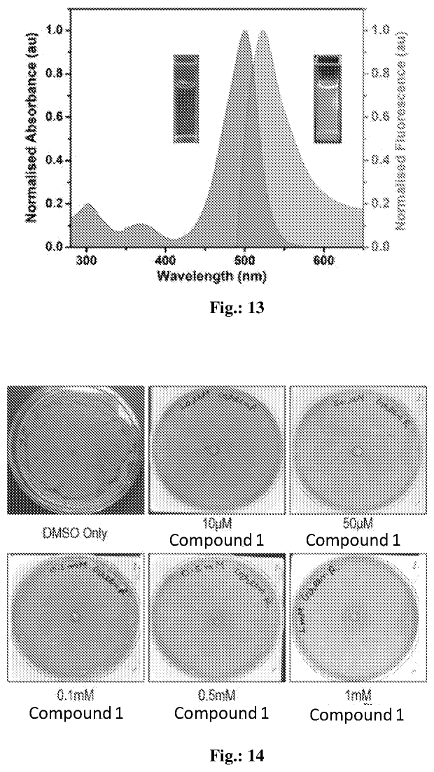

The excitation maximum wavelength (λₘₐₓ) of Compound 1, when bound to nucleic acids, is 495 nm, and its emission maximum wavelength (λₘₐₓ) is 523 nm. These specific wavelengths fall within the green region of the visible light spectrum, and offering convenient and easily distinguishable visualization (FIG. 13).

These special fluorescence properties of Compound 1 make it a highly effective tool for nucleic acid detection and quantification, whether in research or diagnostic applications. Its non-fluorescent nature in the unbound state and its green fluorescence upon binding with nucleic acids provide a high level of sensitivity and specificity in its applications.

### Preparation of Kits

A kit for staining nucleic acids, is prepared by mixing Compound 1 as a nucleic acid gel stain soluble in DMSO for detection of substances, hormones, pathogenic microorganisms, viruses, antibodies, enzymes and nucleic acids. A kit for enhancing RT-PCR efficiency, is prepared by mixing Compound 1 and PCR Master mix consisting of PCR buffer, dNTPs, TaqPolymerase, glycerol and water.

### Usage Instructions for Compound 1 DNA Gel Stain

Incorporation in Agarose Gel Pre-Electrophoresis: Compound 1 DNA Gel Stain is supplied as a 10,000× concentrate in DMSO and is designed to be used in a similar manner to traditional nucleic acid stains like Ethidium bromide. For a standard mini gel, mix 5 μl of Compound 1 stain into 50 ml of agarose solution before pouring the gel into the casting tray. This ensures even distribution of the dye within the gel matrix.

Post-Electrophoresis Staining: Alternatively, Compound 1 can be added to the 1×TAE or TBE buffer used during electrophoresis for post-run visualization of DNA or RNA bands. Add 10 μl of Compound 1 to 500 ml of 1×TAE or TBE buffer, ensuring a thorough mix. After allowing the gel to run, leave the gel in this buffer solution for at least 10 minutes before visualizing on a UV/blue light transilluminator. The dye will bind to nucleic acids in the gel, allowing for visualization under the appropriate light.

RT-PCR Incorporation: Compound 1 may also be incorporated directly into a PCR reaction, where it serves to enhance the efficiency and sensitivity of Real-Time PCR. Add the dye to the reaction at concentrations of less than 0.05×. This low concentration of the stain is sufficient to enable its function without inhibiting the PCR reaction.

In all these applications, the dye's unique properties-non-fluorescent in its unbound state and green fluorescent upon binding to nucleic acids-facilitate clear, precise visualization of nucleic acids, offering an effective tool for nucleic acid detection and quantification in various experimental conditions.

### Advantages of Compound 1 as DNA Gel Stain

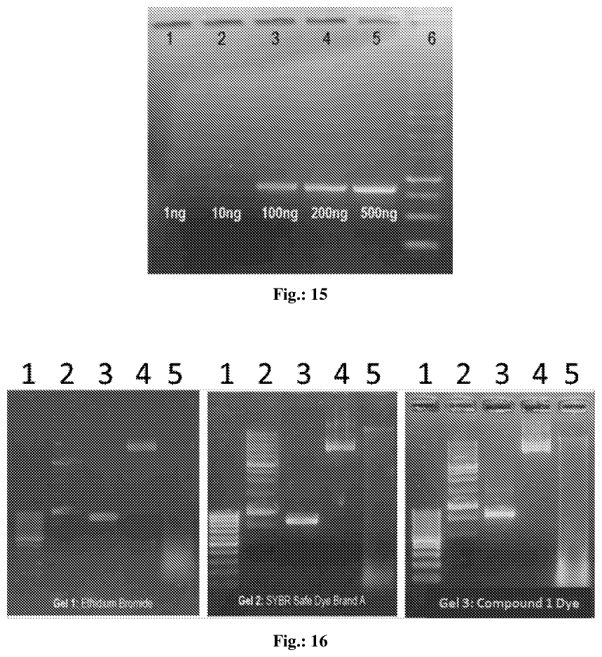

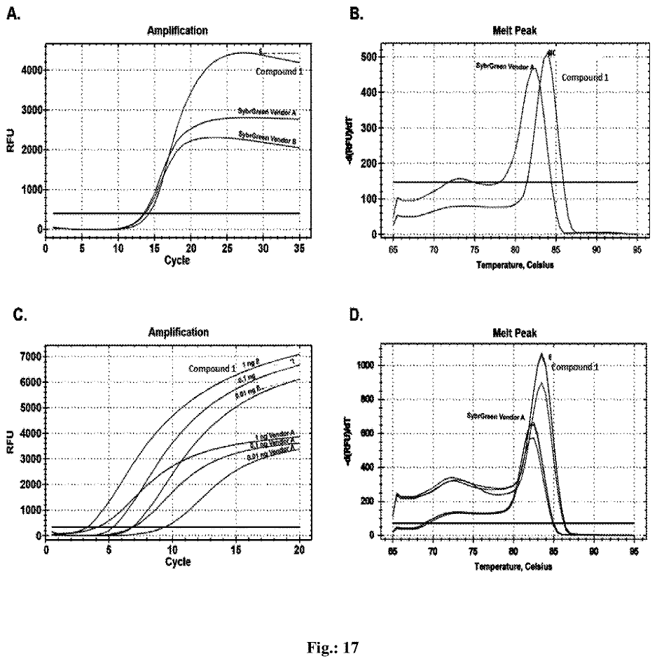

1. Safer Than Ethidium Bromide: Ethidium Bromide, a widely used nucleic acid stain, is known for its mutagenic nature, which raises safety concerns in laboratory settings. In contrast, Compound 1 DNA Gel Stain is non-mutagenic, making it a safer alternative for routine use (FIG. 14).
2. Easy Disposal: Given its non-mutagenic nature, Compound 1 allows for easy and environmentally friendly disposal of gels and used buffers. This reduces the burden of special waste disposal procedures that are necessary with other, more hazardous dyes.
3. No Background Fluorescence: In its unbound state, Compound 1 is non-fluorescent, resulting in no background fluorescence. This property enhances the sensitivity of the dye, as it only fluoresces upon binding with nucleic acids, providing clear and precise visualization.
4. Fluorescent in Blue Light: Compound 1 exhibits fluorescence under blue light illumination. This decreases the exposure risk to harmful UV light that is commonly associated with other nucleic acid stains. It's particularly handy during the elution process from DNA gels, as it allows for safe visualization and extraction of nucleic acids.
5. High Sensitivity: FIG. 15, showcases the sensitivity of Compound 1 as a nucleic acid stain. Different amounts of DNA (1 ng, 10 ng, 100 ng, 200 ng, and 500 ng) were loaded on an agarose gel and stained with Compound 1. The resulting image clearly shows that even as little as 10 ng of DNA can be effectively visualized using Compound 1, highlighting its high sensitivity and effectiveness as a DNA stain. This exceptional sensitivity of Compound 1 makes it a powerful tool for detecting and quantifying even low amounts of DNA in research and diagnostic applications.
6. High Staining Efficiency as compare to other nucleic acid stains: The FIG. 16 presents a comparative study of the staining efficiency of Compound 1 DNA Gel Stain with two other commonly used nucleic acid stains. Three 1% agarose gels loaded with the same nucleic acid samples were stained differently: Gel 1 with Ethidium Bromide, Gel 2 with SYBR Safe dye from a third party, and Gel 3 with Compound 1 DNA Gel Stain. The resulting images provide a visual comparison of the staining efficiency, clarity, and sensitivity of the three different dyes, underscoring the performance of Compound 1 DNA Gel Stain in nucleic acid detection and visualization.

These advantages position Compound 1 as a superior choice for nucleic acid gel staining, offering safer, more reliable, and user-friendly applications in molecular biology.

### Advantages of Compound 1 as Dye in Real-Time PCR

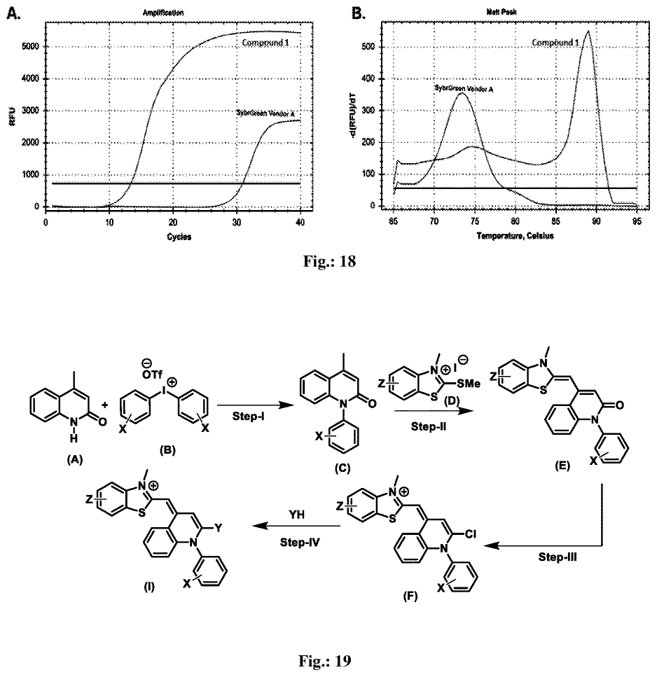

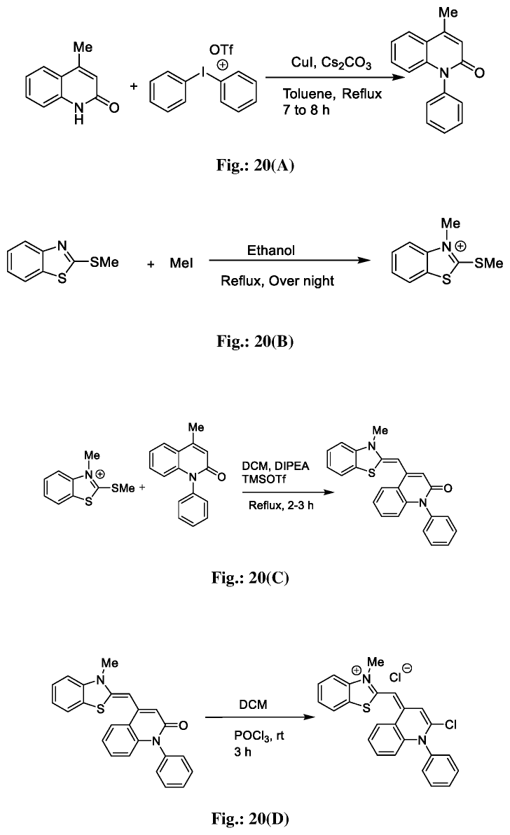

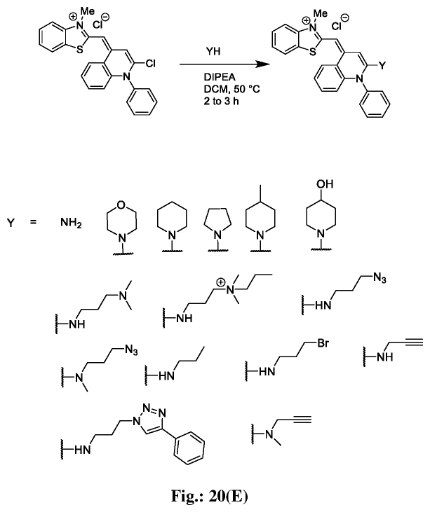

1. Enhanced Sensitivity: Compound 1 exhibits high sensitivity as a dye in Real-Time PCR, allowing for the detection of even low quantities of amplified nucleic acids (FIG. 17). Its ability to provide improved Relative Fluorescence Units (RFU) values compared to other dyes enhances the sensitivity and accuracy of real-time PCR results.
2. Amplification of Larger Targets: Compound 1 offers the unique advantage of enabling the amplification of larger targets in Real-Time PCR. This addresses a significant limitation of existing master mixes, which often struggle to efficiently amplify larger genetic sequences. By facilitating the amplification of larger targets, Compound 1 broadens the range of genetic material that can be effectively detected and analyzed (FIG. 18).
3. Compatibility with Standard PCR Instruments: Compound 1 is compatible with standard real-time PCR instruments, making it a versatile and easily adoptable dye for routine laboratory use. It can be seamlessly incorporated into existing PCR workflows without requiring any specialized equipment or modifications.
4. Safe and Environmentally Friendly: Similar to its application in nucleic acid staining, Compound 1 offers the advantage of being non-mutagenic and safer to handle compared to traditional dyes such as Ethidium Bromide. It does not pose significant health risks to users and does not require special or hazardous waste disposal procedures. This aligns with the increasing emphasis on safety and environmental sustainability in laboratory practices.
5. Cost-Effective Option: Compound 1 provides a cost-effective alternative for real-time PCR applications. Compared to other commercial dyes, Compound 1 offers comparable or even superior performance at a potentially lower cost. This cost-effectiveness makes Compound 1 an attractive choice for laboratories seeking reliable and efficient nucleic acid detection in real-time PCR without compromising their budget.

In summary, Compound 1 demonstrates several advantages as a dye in real-time PCR, including enhanced sensitivity, the ability to amplify larger targets, compatibility with standard instruments, safety, environmental friendliness, and cost-effectiveness. These benefits contribute to improved performance and efficiency in real-time PCR applications, enabling accurate and reliable detection and analysis of nucleic acids in research and diagnostic settings.

### Nucleic Acid Staining Studies:

### Experimental Protocols for Compound 1 DNA Gel Stain:

Staining Nucleic Acids after Electrophoresis:

Place the gel in a suitable container and immerse it in the Compound 1 DNA Gel Stain solution. If using the Compound 1 concentrate (10,000×), dilute it in the appropriate buffer (e.g., TAE or TBE) before use. Ensure the gel is fully immersed by adding enough Compound 1 DNA Gel Stain solution to cover the gel. For standard minigels, a volume of 50 mL is typically sufficient. Adjust the volume of staining solution accordingly for larger gels.

Incubate the gel for 30 minutes, ensuring it is covered with aluminum foil or kept in the dark to protect it from light. Agitate the gel continuously using an orbital shaker set at 50 rpm. No destaining step is required.

### Precasting Compound 1 DNA Gel Stain in Agarose Gels:

Prepare the agarose gel by directly incorporating Compound 1 DNA Gel Stain. Compound 1 DNA Gel Stain is provided in a buffer form, so replace the buffer with the Compound 1 DNA Gel Stain when preparing the agarose gel. If using the 10,000× Compound 1 concentrate, dilute it 1:10,000 in the agarose gel buffer (e.g., 1×TBE or 1×TAE). Add the buffer-stain mixture to the powdered agarose. For instance, if you need 30 mL of molten agarose for your tray and are using TBE buffer, mix 3 μL of the 10,000× Compound 1 concentrate with 30 mL of 1×TBE, and add it to the powdered agarose. Thoroughly mix the solution.

Note: Gels containing Compound 1 DNA Gel Stain may exhibit slightly slower mobility of nucleic acid fragments compared to gels without stain when using precast gels with ethidium bromide.

Run the gel using an appropriate running buffer compatible with Compound 1 DNA Gel Stain. No additional staining or destaining steps are necessary.

### Viewing and Photographing the Gel:

### To Visualize Stained Gels and Capture Images:

Use a standard 300 nm transilluminator or a 254 nm epi- or transilluminator.

Employ a blue-light transilluminator, such as the Safe Imager™ 2.0 Blue-Light transilluminator (Cat. no. G6600), for optimal visualization of DNA stained with Compound 1. Utilize imaging systems equipped with UV excitation sources or excitation between 470-530 nm.

Note: When excising gel bands stained with Compound 1 for ligation reactions, it is recommended to use a blue-light source (e.g., Safe Imager™ 2.0 Blue-Light transilluminator) instead of UV light. The combination of UV light sources with Compound 1 DNA Gel Stain may potentially reduce cloning efficiencies.

For photography purposes, consider using Polaroid™ 667 black-and-white print film with a Compound 1 photographic filter (Cat. no. S37100), SYPRO™ photographic filter (Cat. no. S6656), or a Kodak™ Wratten #9 filter. These filters provide a similar detection sensitivity to ethidium bromide when working with Compound 1 DNA Gel Stain. Avoid using a standard ethidium bromide photographic filter with Compound 1 DNA Gel Stain. Alternatively, gels stained with Compound 1 can be imaged using a CCD camera or a laser-based scanner.

We claim:

1. A compound of formula I, nucleic acid conjugates, complexes and salts thereof, 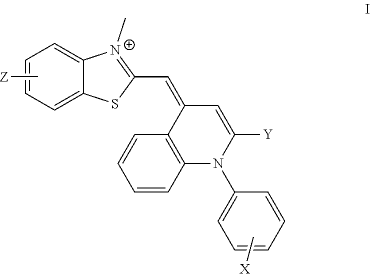
wherein;
X, Z are independently selected from the group consisting of hydrogen, hydroxy, alkyl (C1-C5), alkoxy (OC1-OC5), amine and halogen;
Y is independently selected from the group consisting of

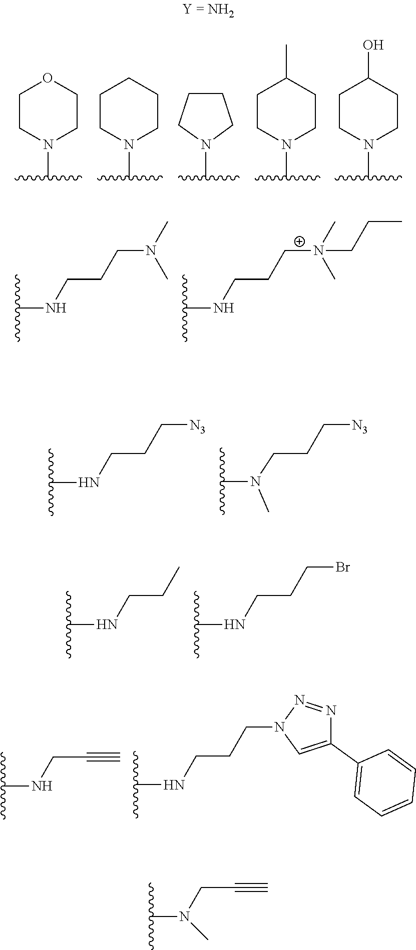

2. The compound as claimed in claim 1, wherein the representative compounds are selected from the group consisting of:
i. 3-methyl-2-((2-morpholino-1-phenylquinolin-4(1H)-ylidene)methyl)benzo[d]thiazol-3-ium;
ii. 2-((2-((3-azidopropyl)amino)-1-phenylquinolin-4(1H)-ylidene)methyl)-3-methylbenzo[d]thiazol-3-ium;
iii. 2-((2-((3-(dimethylamino)propyl)amino)-1-phenylquinolin-4(1H)-ylidene)methyl)-3-methylbenzo[d]thiazol-3-ium;
iv. 2-((2-((3-azidopropyl)(methyl)amino)-1-phenylquinolin-4(1H)-ylidene)methyl)-3-methylbenzo[d]thiazol-3-ium;
v. 3-methyl-2-((1-phenyl-2-(propylamino)quinolin-4(1-ylidene)methyl)benzo[d]thiazol-3-ium;
vi. 3-methyl-2-((2-(methyl(prop-2-yn-1-yl)amino)-1-phenylquinolin-4(1H)-ylidene)methyl)benzo[d]thiazol-3-ium;
vii. 3-methyl-2-((1-phenyl-2-(prop-2-yn-1-ylamino)quinolin-4(1H)-ylidene)methyl)benzo[d]thiazol-3-ium;
viii. 2-((2-((1-(dimethyl(propyl)-14-azaneyl)propan-1-ylium-3-yl)amino)-1-phenylquinolin-4(1H)-ylidene)methyl)-3-methylbenzo[d]thiazol-3-ium;
ix. 2-((2-amino-1-phenylquinolin-4(1H)-ylidene)methyl)-3-methylbenzo[d]thiazol-3-ium;
x. 2-((2-(4-hydroxypiperidin-1-yl)-1-phenylquinolin-4(1H)-ylidene)methyl)-3-methylbenzo[d]thiazol-3-ium;
xi. 3-methyl-2-((1-phenyl-2-(piperidin-1-yl)quinolin-4(1H)-ylidene)methyl)benzo[d]thiazol-3-ium;
xii. 3-methyl-2-((1-phenyl-2-((3-(4-phenyl-JH-1,2,3-triazol-1-yl)propyl)amino)quinolin-4(1H)-ylidene)methyl)benzo[d]thiazol-3-ium;
xiii. 5-hydroxy-3-methyl-2-((2-morpholino-1-phenylquinolin-4(1H)-ylidene)methyl)benzo[d]thiazol-3-ium;
xiv. 3,5-dimethyl-2-((2-morpholino-1-phenylquinolin-4(1H)-ylidene)methyl)benzo[d]thiazol-3-ium;
xv. 4-chloro-3-methyl-2-((2-morpholino-1-phenylquinolin-4(1H)-ylidene)methyl)benzo[d]thiazol-3-ium;
xvi. 7-chloro-3-methyl-2-((2-morpholino-1-phenylquinolin-4(1H)-ylidene)methyl)benzo[d]thiazol-3-ium; and
xvii. 6-chloro-3-methyl-2-((2-morpholino-1-phenylquinolin-4(1H)-ylidene)methyl)benzo[d]thiazol-3-ium.

3. A process for the preparation of compound of general formula I, comprising the steps of: 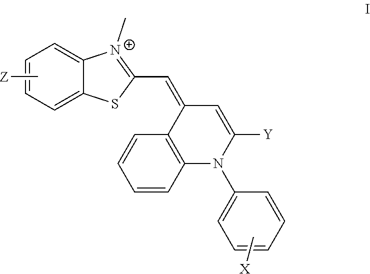
wherein;
X, Z are independently selected from the group consisting of hydrogen, hydroxy, alkyl (C1-C5), alkoxy (OC1-OC5), amine and halogen;
Y is independently selected from the group consisting of 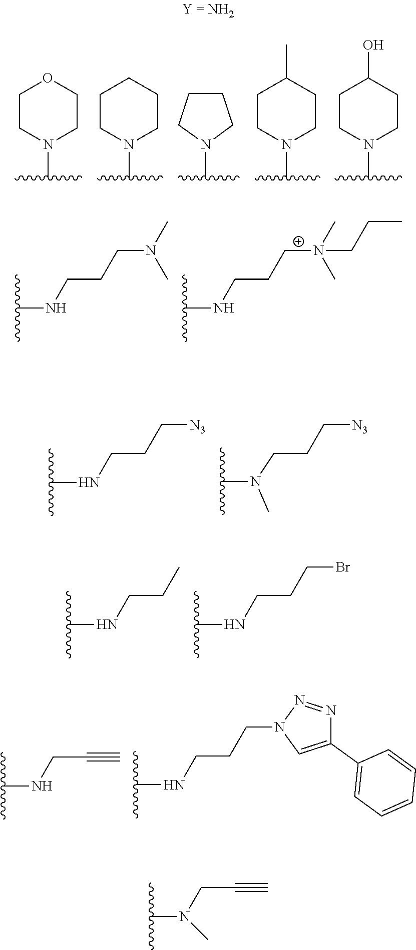
wherein said method comprising the steps of;
a. reacting a mixture of 4-methylquinolin-2(1H)-one (A) and substituted diphenyliodonium triflate (B) in the presence of a copper salt and a base cesium carbonate in presence of an organic solvent under room temperature to reflux condition to form a compound of formula (C) 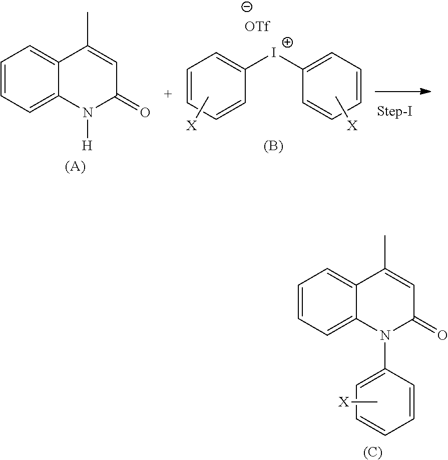
wherein X is as defined above;
b. reacting a cooled mixture of the compound (C) as obtained in step (a) and substituted 3-methyl-2-(methylthio)benzo[d]thiazol-3-ium (D), in the presence of N,N-diisopropylethylamine in presence of an organic solvent followed by addition of trimethysilyl trifluoromethanesulfonate dropwise to the solution which is then refluxed for 30 min to 24 hr to form a compound of formula (E) 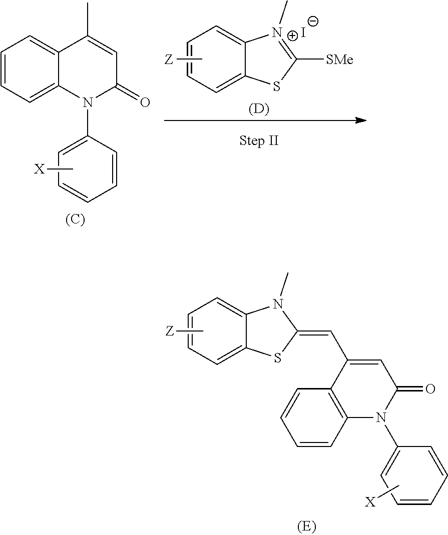
wherein X and Z are as defined above.
c. reacting the compound (E) as obtained in step (b) in dry dichloromethane with a solution of POCl₃ under reflux to form a compound of formula (F) 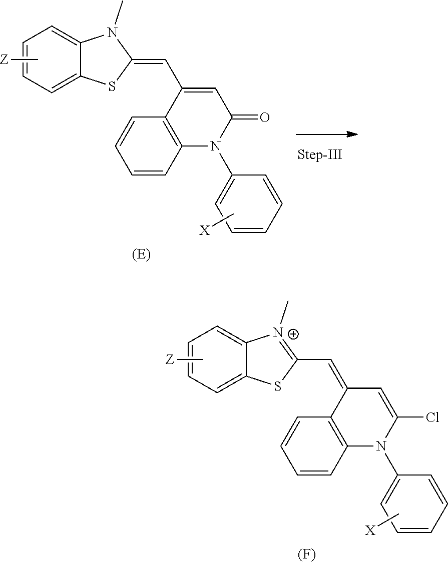
wherein X and Z are as defined above; and
d. reacting the compound (F) as obtained in step (c) with substituted amines (YH) in presence of an organic solvent, and
isolating the compound of formula I from the reaction mixture and purifying by washing with organic solvents or by chromatography, 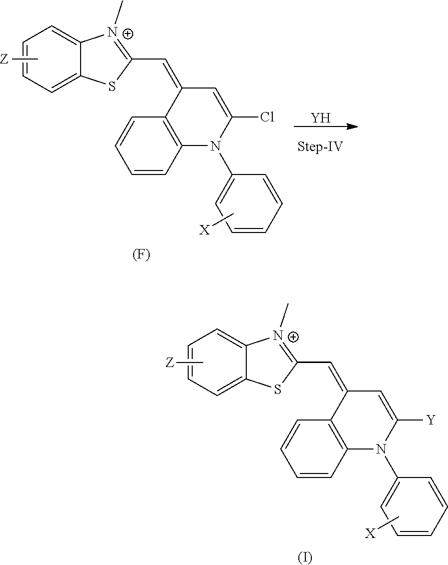
wherein X, Y and Z are as defined above.

4. The process as claimed in claim 3, wherein the organic solvent is selected from the solvent group consisting of toluene, dichloromethane, dichloroethane, acetonitrile, dimethylsulphoxide, dimethylformamide, water, methanol, ethyl acetate, hexane and tetrahydrofuran.

5. The compounds as claimed in claim 1, wherein the compound is for use in
analyzing nucleic acids (DNA, RNA), and other biological substances of diagnostic importance;
developing of diagnostic kit for detection of substances, hormones, pathogenic microorganisms, viruses, antibodies, enzymes and nucleic acids, particularly those implicated in disease states and,
for preparing of fluorescent probes, tags, markers, diagnostics, ion sensor, pharmaceuticals for detecting/trapping ions in fluorescence-based imaging and/or analysis of cells, biological fluids and chemical mixture.

6. A kit for staining nucleic acids, comprising the compound as claimed in claim 1 as a nucleic acid gel stain preferable with DMSO for detection of substances, hormones, pathogenic microorganisms, viruses, antibodies, enzymes and nucleic acids.

7. A kit for enhancing RT-PCR, comprising the compound as claimed in claim 1 and PCR Master mix consisting of PCR buffer, dNTPs, TaqPolymerase, glycerol and water.

8. The nucleic acid gel stain as claimed in claims 6, 7, wherein the nucleic acid gel stain exhibiting dual functionality in nucleic acid visualization and enhancing Real-Time Polymerase Chain Reaction (RT-PCR) processes, enhanced sensitivity in RT-PCR processes and amplification of larger targets in RT-PCR processes, thereby expanding the range of sequences which are efficiently amplified and detected.

9. The compounds as claimed in claim 1 and 2, wherein the compound exhibits affinity towards nucleic acids selected from the group consisting of genomic DNA, PCR products, plasmids, and RNA.

10. The nucleic acid gel stain as claimed in claims 6, 7, wherein said gel stain has sensitivity for less than 10 ng of DNA.

11. The nucleic acid gel stain as claimed in claims 6, 7, wherein the stain is free of hazardous material.

12. A method of staining nucleic acids using the compounds as claimed in claim 1, comprising the steps of: (a) mixing a sample containing nucleic acids with the compounds of claims 1 and 2; and (b) exposing said sample to blue light or UV excitation to visualize the nucleic acids.

13. A method for enhancing RT-PCR processes using the compounds as claimed in claims 1 and 2, comprising the steps of: (a) incorporating the compounds of claims 1 and 2 into an RT-PCR master mix; and (b) performing RT-PCR, resulting in enhanced sensitivity and the ability to amplify larger targets.
